(19) United States
(12) Patent Application Publication
(10) Pub. No.: US 2025/0262033 A1
(43) Pub. Date: Aug. 21, 2025

(54) SOCKET FORMING TOOLS FOR AUTOTRANSPLANTATION

(71) Applicant: Form and Function Dental Services, P.C., Wynnewood, PA (US)

(72) Inventor: Dumitru Gogarnoiu, Wynnewood, PA (US)

(21) Appl. No.: 18/582,186

(22) Filed: Feb. 20, 2024

(51) Int. Cl. A61C 8/00; A61B 6/51; A61C 13/00; A61L 27/36; A61L 31/02

(52) U.S. Cl. CPC A61C 8/0089; A61C 13/0004; A61C 13/0013; A61L 27/3608; A61L 27/365; A61L 31/026; A61B 6/51; A61L 2430/12

(57) ABSTRACT

A socket preparation tool configured to facilitate autotransplantation of a donor tooth to a recipient site is provided. The socket preparation tool includes a tooth replica of the donor tooth. A coronal portion is optionally included for facilitating alignment of the tooth replica adjacent teeth during a simulated autotransplantation. A radicular portion includes a smooth surface configured to facilitate compression of soft bone or soft tissue of the tooth replica or a contoured surface adapted for filing or cutting bone of the tooth replica. A cylindrical attachment is configured to be coupled to the tooth replica, such as via a receiver. A method of guided autotransplantation of a donor tooth via the customized socket preparation tool includes at least fitting and adjusting the socket preparation tool coupled to the tooth replica in the recipient site, before extracting the donor tooth.

### BACKGROUND OF THE INVENTION

The replacement of a lost or compromised tooth can be achieved through dental implant, fixed partial denture, or tooth autotransplantation. Autotransplantation is the surgical transfer of a tooth from its original location to a receiving socket of the same patient. This technique offers several advantages over other treatment options (e.g., dental implants or fixed partial prosthesis). For example, autotransplantation helps maintain the periodontal ligament (PDL) and the surrounding bone, allows for the formation of biologic width supra-crestally, and results in better aesthetics compared to dental implants, for example, which establish biologic width sub-crestally and so have a less aesthetic appearance due to the metallic grey color showing through the gum tissue. In general, autotransplantation is considered to be one of the most biologic techniques for replacing a missing tooth.

In conventional processes for autotransplantation of teeth, the donor teeth serve as the only guide for preparing new sockets at recipient positions. As a result, the donor teeth need to be extracted early and placed into new sockets multiple times until they a proper fit is achieved. This prolonged extra-alveolar time and resulting damage to PDL cells from multiple fitting attempts can have a negative impact on the success of autotransplantation.

To attempt to compensate for these problems, rapid prototyping replicas based on cone-beam computed tomography (CBCT) data have been used as alternatives to donor teeth for preparing new sockets. In this way, the extra-alveolar time can be reduced and injury to PDL cells (as compared to conventional processes) is minimized. This is achieved because the replica is used as a guide prior to the extraction of the donor tooth. Nevertheless, multiple fittings are still required to ensure accurate 3D fitting of the replica with the shape of the donor teeth roots. Thus, improvements in processes related to autotransplantation is desired, particularly for reducing extra-alveolar time, facilitating a passive adaptability for the donor tooth through bone contouring, maintaining the periodontal membrane and pulp vitality, or mitigating or preventing inflammatory resorption or ankylosis.

### SUMMARY OF THE INVENTION

According to one aspect of the invention, a method of guided autotransplantation of a donor tooth via a customized socket preparation tool is provided. The method includes a step of (a) preparing a three-dimensional tooth replica of the donor tooth via cone-beam based tomography (CBCT) imaging. Step (b) involves constructing a digital model for characterizing the tooth replica by segmenting selected images of the tooth replica. Step (c) includes converting the selected segmented images to stereo lithography (STL) images. Further, step (d) involves transmitting the STL files of the tooth replica to a computer aided manufacturing process for fabricating the tooth replica. In addition, step (e) includes selecting and transmitting STL files of the customized socket preparation tool to a computer aided manufacturing process for fabricating the socket preparation tool. The method also includes a step (f) of selecting the socket preparation tool and coupling the socket preparation tool to the tooth replica. Still further, step (g) includes fitting and adjusting the socket preparation tool coupled to the tooth replica in the recipient site. Finally, after step (g), step (h) includes extracting the donor tooth.

According to another aspect of the invention, a socket preparation tool configured to facilitate autotransplantation of a donor tooth to a recipient site is provided. The socket preparation tool includes a tooth replica of the donor tooth; a coronal portion for facilitating alignment of the tooth replica adjacent teeth during a simulated autotransplantation; a radicular portion comprising a smooth surface configured to facilitate compression of soft bone of the tooth replica during a simulated autotransplantation; and a cylindrical attachment configured to be coupled to the tooth replica, the attachment comprising a slot.

According to still another aspect of the invention, a socket preparation tool configured to facilitate autotransplantation of a donor tooth to a recipient site is provided. The socket preparation tool includes a tooth replica of the donor tooth; a coronal portion for facilitating alignment of the tooth replica adjacent teeth during a simulated autotransplantation; a radicular portion comprising a contoured surface adapted for filing bone of the tooth replica during a simulated autotransplantation; and a cylindrical attachment configured to be coupled to the tooth replica, the attachment comprising a slot.

According to yet another aspect of the invention, a socket preparation tool configured to facilitate autotransplantation of a donor tooth to a recipient site is provided. The socket preparation tool includes a tooth replica of the donor tooth; a radicular portion comprising a smooth surface configured to facilitate compression of soft tissue during a simulated autotransplantation; and a cylindrical attachment configured to be coupled to the tooth replica, the attachment comprising a slot.

According to yet another aspect of the invention, a socket preparation tool configured to facilitate autotransplantation of a donor tooth to a recipient site is provided. The socket preparation tool includes a tooth replica of the donor tooth; a radicular portion comprising a contoured surface adapted for cutting bone during a simulated autotransplantation; and a cylindrical attachment configured to be coupled to the tooth replica, the attachment comprising a slot.

### BRIEF DESCRIPTION OF THE DRAWINGS

The foregoing summary, as well as the following detailed description of desired embodiments of the invention, will be better understood when read in conjunction with the appended drawings, which are incorporated herein and constitute part of this specification. For the purposes of illustrating the invention, there are shown in the drawings embodiments that are presently desired. It should be understood, however, that the invention is not limited to the precise arrangements and instrumentalities shown. In the drawings, the same reference numerals are employed for designating the same elements throughout the several figures. In the drawings:

FIG. 1 depicts a three-dimensional tooth replica of the donor tooth according to an exemplary embodiment of the present invention;

FIG. 2A is a facial view of a socket preparation tool according to a first embodiment of the present invention;

FIG. 2B is a lingual view of the socket preparation tool of FIG. 2A;

FIG. 2C is a mesial view of the socket preparation tool of FIG. 2A;

FIG. 2D is a distal view of the socket preparation tool of FIG. 2A;

FIG. 2E is an occlusal view of the socket preparation tool of FIG. 2A;

FIG. 2F is an apical view of the socket preparation tool of FIG. 2A;

FIG. 3A is a facial view of a socket preparation tool according to a second embodiment of the present invention;

FIG. 3B is a lingual view of the socket preparation tool of FIG. 3A;

FIG. 3C is a mesial view of the socket preparation tool of FIG. 3A;

FIG. 3D is a distal view of the socket preparation tool of FIG. 3A;

FIG. 3E is an apical view of the socket preparation tool of FIG. 3A;

FIG. 4A is a facial view of a socket preparation tool according to a third embodiment of the present invention;

FIG. 4B is a mesial view of the socket preparation tool of FIG. 4A;

FIG. 4C is a perspective view of the socket preparation tool of FIG. 4A;

FIG. 4D is an occlusal view of the socket preparation tool of FIG. 4A;

FIG. 4E is an apical view of the socket preparation tool of FIG. 4A;

FIG. 5A is a facial view of a socket preparation tool according to a fourth embodiment of the present invention;

FIG. 5B is a mesial view of the socket preparation tool of FIG. 5A;

FIG. 5C is an occlusal view of the socket preparation tool of FIG. 5A;

FIG. 5D is an apical view of the socket preparation tool of FIG. 5A;

FIGS. 6A-6D depict views of a socket preparation tool in varying sizes according to exemplary embodiments of the present invention; and

FIG. 7 is a flow chart illustrating the steps performed for guided autotransplantation of a donor tooth via the customized socket preparation tool of FIGS. 2A-6D into a patient.

### DETAILED DESCRIPTION OF THE INVENTION

Certain terminology is used herein for convenience only and is not to be taken as a limitation on the present invention. The terminology includes the words specifically mentioned, derivatives thereof and words of similar import. The term “facial” is defined to mean a direction closer to the lips and cheek of the user. The term “lingual” is defined to mean a direction closer to the tongue of the user. The term “mesial” is defined to mean a direction closer to an imaginary centerline of the mouth of the user. The term “distal” is defined to mean a direction farther from the imaginary centerline of the mouth. The term “occlusal” is defined to mean the top surface, such as the chewing surface, of a tooth. Further, as used herein, the term “configuration” is defined to mean size and/or shape. The following describes desired embodiments of the invention. However, it should be understood based on this disclosure, that the invention is not limited by the desired embodiments of the invention.

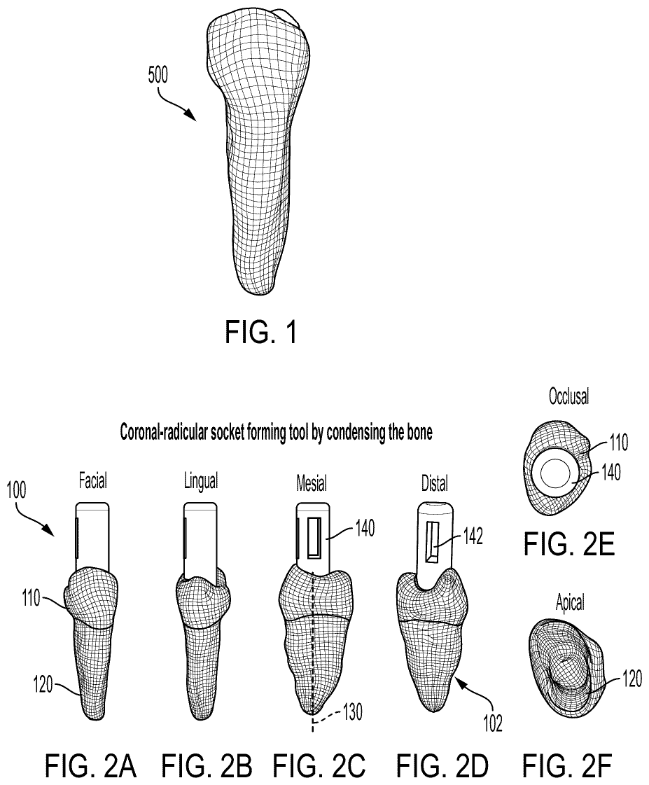

Referring generally to the figures, several embodiments of a socket preparation tool according to the present invention are shown. FIG. 1 depicts a three-dimensional tooth replica 500 of a donor tooth according to an exemplary embodiment of the present invention. The purpose of this tooth replica 500 is to continuously monitor the socket formation process, as well as the relationship with neighboring teeth without compromising occlusion. The donor tooth is generally selected from the same quadrant as the neighboring teeth (for similar morphological features), but is not required and the donor tooth is not limited to what is illustrated in the figures.

FIGS. 2A-2F show six different views of a socket preparation tool 100 according to a first embodiment of the present invention. FIG. 2A is a facial view; FIG. 2B is a lingual view; FIG. 2C is a mesial view; FIG. 2D is a distal view; FIG. 2E is an occlusal view; and FIG. 2F is an apical view of socket preparation tool 100.

Referring to any of FIGS. 2A-2F, socket preparation tool 100 is configured to facilitate autotransplantation of a donor tooth to a recipient site. The tool 100 includes a tooth replica of the donor tooth, such as tooth replica 500 (FIG. 1). The tool 100 includes a body 102 having a coronal portion 110, a radicular portion 120, and a longitudinal axis 130 extending between coronal portion 110 and radicular portion 120. In an exemplary embodiment, the coronal portion 110 is configured for facilitating alignment of the tooth replica 500 adjacent teeth during a simulated autotransplantation. The radicular portion 120 includes a smooth surface (e.g., relative to a surface of another component of the tool 100, such as the coronal portion 110 or the cylindrical attachment 140) configured to facilitate compression of soft bone of the tooth replica 500 during a simulated autotransplantation. The contours and geometry of the coronal portion 110 and radicular portion 120 illustrated in FIGS. 2A-2F is not intended to be limiting and is dependent, in part, by one or more morphological characteristics of the donor tooth and/or the physiological characteristics of the patient.

A cylindrical attachment 140 is configured to be coupled to the tooth replica 500. Additionally or optionally, the attachment 140 comprising a receiver 142, such as a slot. The receiver 142 is configured to be directly or indirectly coupled to a driver, such as driver 600 (FIG. 7). The driver 600 is configured to drive or actuate the socket preparation tool or socket forming tool 100 in at least one direction (e.g., clockwise or counterclockwise, forward or backward, etc.) in order to adjust the fit of the socket preparation tool 100 in the recipient site.

Still further, one skilled in the art would understand from the description herein that the socket preparation tool 100 can be customized to accommodate a form factor of the tooth replica 500 or a spectrum of form factors of the tooth replica 500 within an expected range of value based on the donor tooth. For example, the radicular portion 120 of the socket preparation tool 100 is adjustable to accommodate the form factor or the spectrum of form factors desired in an autotransplantation process.

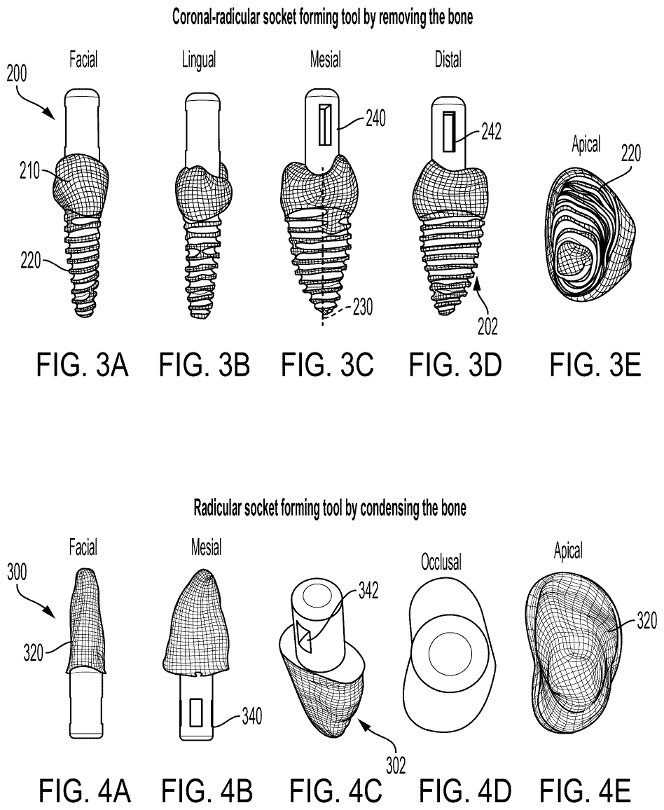

FIGS. 3A-3E show five different views of a socket preparation tool 200 according to a second embodiment of the present invention. FIG. 3A is a facial view; FIG. 3B is a lingual view; FIG. 3C is a mesial view; FIG. 3D is a distal view; and FIG. 3E is an apical view of socket preparation tool 200. In general, this second embodiment is similar to the other embodiments discussed throughout the specification, but differs in some respects.

Referring to any of FIGS. 3A-3E, socket preparation tool 200 is configured to facilitate autotransplantation of the donor tooth to the recipient site. The tool 100 includes a tooth replica of the donor tooth, such as tooth replica 500 (FIG. 1). The tool 100 includes a body 202 having a coronal portion 210, a radicular portion 220, and a longitudinal axis 230 extending between coronal portion 210 and radicular portion 220. As in the first embodiment discussed above, for example, the coronal portion 210 is configured for facilitating alignment of the tooth replica 500 adjacent teeth during a simulated autotransplantation. Likewise, a cylindrical attachment 240 is configured to be coupled to the tooth replica 500, and the attachment 240 has a receiver 242. Characteristics of the receiver 242 and attachment 240 are similar to those described with respect to attachment 140 and receiver 142 discussed above.

However, unlike the first embodiment discussed above, for example, the radicular portion 220 includes a non-smooth or contoured surface. In an exemplary embodiment, the non-smooth or contoured surface of the radicular portion 220 is adapted for filing bone of the tooth replica 500 during a simulated autotransplantation. The geometry of the contoured surface of the radicular portion 220 illustrated in FIGS. 3a-3e is not intended to be limiting and is dependent, in part, by one or more morphological characteristics of the donor tooth and/or the physiological characteristics of the patient.

Additionally, as discussed in the first embodiment above, one skilled in the art would understand from the description herein that the socket preparation tool 200 can be customized to accommodate a form factor of the tooth replica 500 or a spectrum of form factors of the tooth replica 500 within an expected range of value based on the donor tooth. For example, the radicular portion 220 of the socket preparation tool 200 is adjustable to accommodate the form factor or the spectrum of form factors desired in an autotransplantation process.

FIGS. 4A-4E show five different views of a socket preparation tool 300 according to a third embodiment of the present invention. FIG. 4A is a facial view; FIG. 4B is a mesial view; FIG. 4C is a perspective view; FIG. 4D is an occlusal view; and FIG. 4E is an apical view of socket preparation tool 300. In general, this third embodiment is similar to the other embodiments discussed throughout the specification, but differs in some respects.

Referring to any of FIGS. 4A-4E, socket preparation tool 300 is configured to facilitate autotransplantation of the donor tooth to the recipient site. The tool 300 includes a tooth replica of the donor tooth, such as tooth replica 500 (FIG. 1). Unlike the first and second embodiments, the tool 300 includes a body 302 having radicular portion 320 only. Similar to the first embodiment discussed above, for example, the radicular portion 320 defines a smooth surface configured to facilitate compression of soft tissue during a simulated autotransplantation. The geometry of the radicular portion 320 illustrated in FIGS. 4a-4e is not intended to be limiting and is dependent, in part, by one or more morphological characteristics of the donor tooth and/or the physiological characteristics of the patient.

Additionally, as discussed in the embodiments above, a cylindrical attachment 340 is configured to be coupled to the tooth replica 500. Additionally or optionally, the attachment 340 comprising a receiver 342. The receiver 342 is configured to be directly or indirectly coupled to a driver 600 (FIG. 7). The driver 600 is configured to drive or actuate the socket preparation tool or socket forming tool 100 in at least one direction (e.g., clockwise or counterclockwise, forward or backward, etc.) in order to adjust the fit of the socket preparation tool 300 in the recipient site.

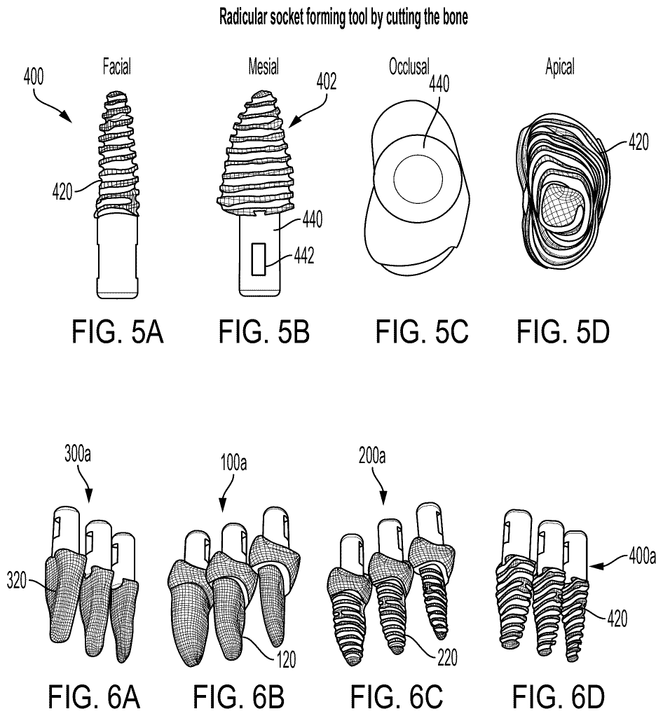

FIGS. 5A-5D show four different views of a socket preparation tool 400 according to a fourth embodiment of the present invention. FIG. 5A is a facial view; FIG. 5B is a mesial view; FIG. 5C is an occlusal view; and FIG. 5D is an apical view of socket preparation tool 400. In general, this fourth embodiment is similar to the other embodiments discussed throughout the specification, but differs in some respects.

Referring to any of FIGS. 5A-5D, socket preparation tool 400 is configured to facilitate autotransplantation of the donor tooth to the recipient site. The tool 400 includes a tooth replica of the donor tooth, such as tooth replica 500 (FIG. 1). Unlike the first and second embodiments, the tool 400 includes a body 402 having radicular portion 420 only. Similar to the second embodiment discussed above, for example, the radicular portion 420 includes a non-smooth or contoured surface. In an exemplary embodiment, the non-smooth or contoured surface of the radicular portion 420 is adapted for cutting bone of the tooth replica 500 during a simulated autotransplantation. The geometry of the contoured surface of the radicular portion 420 illustrated in FIGS. 5A-5D is not intended to be limiting and is dependent, in part, by one or more morphological characteristics of the donor tooth and/or the physiological characteristics of the patient.

Additionally, as discussed in the embodiments above, a cylindrical attachment 440 is configured to be coupled to the tooth replica 500. Additionally or optionally, the attachment 440 comprising a receiver 442. The receiver 442 is configured to be directly or indirectly coupled to a driver 600 (FIG. 7). The driver 600 is configured to drive or actuate the socket preparation tool or socket forming tool 400 in at least one direction (e.g., clockwise or counterclockwise, forward or backward, etc.) in order to adjust the fit of the socket preparation tool 400 in the recipient site.

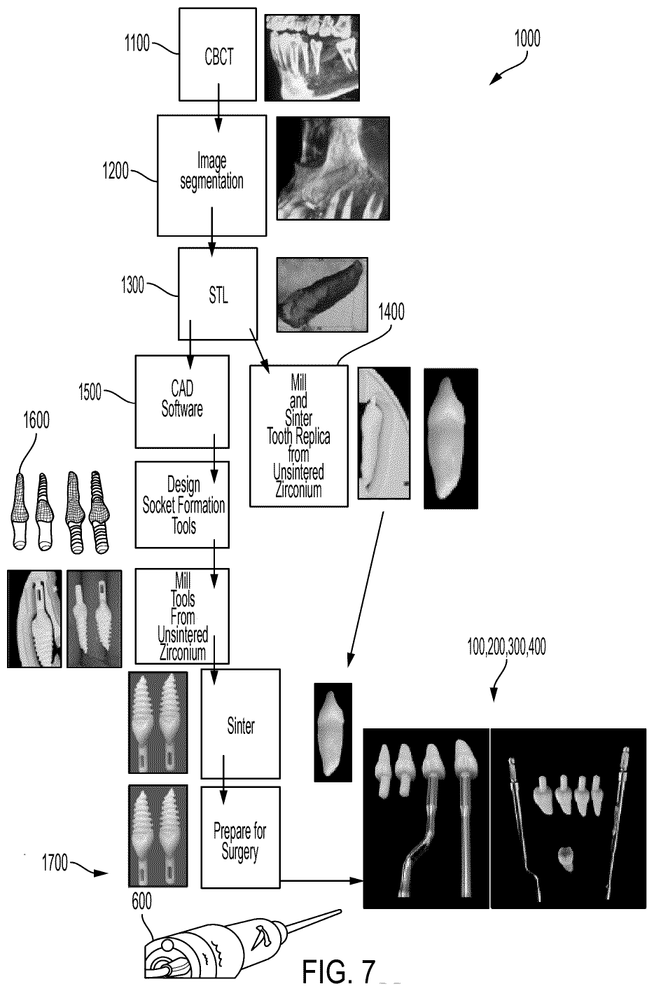

FIGS. 6A-6D depict view of a socket preparation tool in varying sizes according to exemplary embodiments of the present invention. FIG. 6A depicts exemplary embodiments of socket preparation tool 300a, which illustrates varying sizes and geometry of the radicular portion 320 of the socket preparation tool 300. FIG. 6B depicts exemplary embodiments of socket preparation tool 100a, which illustrates varying sizes and geometry of at least the radicular portion 120 of the socket preparation tool 100. FIG. 6C depicts exemplary embodiments of socket preparation tool 200a, which illustrates varying sizes and geometry of at least the radicular portion 220 of the socket preparation tool 200. FIG. 6D depicts exemplary embodiments of socket preparation tool 400a, which illustrates varying sizes and geometry of the radicular portion 420 of the socket preparation tool 400.

In this way, the socket preparation tool can be “custom fit” or “customized.” As used herein and throughout the specification, the term “custom fit” is not limited to a user-by-user (e.g. patient) basis, but can also refer to providing a relatively smaller number of standard incremental sizes (S, M, L, XL, 2XL) of the socket preparation tool. The socket preparation tool of a standard incremental size (e.g. XS) may provide additional customization (or further custom fit) by including at least one adjustable component (e.g. a radicular portion and/or coronal portion, etc.). One skilled in the art would understand from the description herein that the adjustable component may be configured for permitting adjustments that result in more individualized or custom fit.

Referring now to FIG. 7, a method of guided autotransplantation of a donor tooth via a customized socket preparation tool, such as any of the socket preparation tools 100, 200, 300, 400 described above, is detailed herein. The method 1000 includes a step 1100 of (a) preparing a three-dimensional tooth replica (e.g. tooth replica 500 of FIG. 1) of the donor tooth via cone-beam based tomography (CBCT) imaging. Then, in step 1200, (b) a digital model is constructed for characterizing the tooth replica 500 by segmenting selected images of the tooth replica 500. Next, step 1300 includes (c) converting the selected segmented images to stereo lithography (STL) images and step 1400 involves (d) transmitting the STL files of the tooth replica to a computer aided manufacturing process for fabricating the tooth replica 500. In step 1500, the method includes (e) selecting and transmitting STL files of the customized socket preparation tool 100, 200, 300, 400 to a computer aided manufacturing process for fabricating the socket preparation tool 100, 200, 300, 400. In step 1600, (f) the socket preparation tool 100, 200, 300, 400 is selected and coupled to the tooth replica 500. The method includes step 1700, which involves (g) fitting and adjusting the socket preparation tool coupled to the tooth replica in the recipient site. Then, in step (h) after performance of step 1700, the donor tooth is extracted.

Advantageously, with the use of the socket preparation tool 100, 200, 300, 400, extra-alveolar time is reduced, a passive adaptability for the donor tooth through bone contouring is facilitated, the periodontal membrane and pulp vitality is maintained, and/or inflammatory resorption or ankylosis is mitigated or prevented. This is achieved because the adjustments in fit can be made with socket preparation tool 100, 200, 300, 400 with tooth replica 500 prior to extraction of donor tooth and ultimate transplant of the donor tooth in the recipient site.

In exemplary embodiments of the method 1000, steps 1400 and 1500 comprising steps (d) and (e) include substeps of milling the tooth replica 500 or the socket preparation tool 100, 200, 300, 400 from unsintered zirconium block. Additionally or optionally, method 1000 further includes a step (i) of driving the socket forming tool 100, 200, 300, 400 in at least one direction via a driver (e.g. driver 600 in FIG. 7) configured to be directly or indirectly coupled to the socket forming tool 100, 200, 300, 400. In one non-limiting example this attachment is facilitated via a receiver (e.g. receivers 142, 242, 342, 442). Still further, method 1000 further includes step (j) of fitting the donor tooth in the recipient site (e.g., into a predetermined implant location in a mouth of the patient). To facilitate this fit, the customized socket preparation tool 100, 200, 300, 400 is selected to accommodate a form factor of the tooth replica or a spectrum of form factors of the tooth replica 500 within an expected range of values based on the donor tooth.

Although the invention is illustrated and described herein with reference to specific embodiments, the invention is not intended to be limited to the details shown. Rather, various modifications may be made in the details within the scope and range of equivalents of 10 the claims and without departing from the invention.

What is claimed:

1. A method of guided autotransplantation of a donor tooth via a customized socket preparation tool, the method comprising:
(a) preparing a three-dimensional tooth replica of the donor tooth via cone-beam based tomography (CBCT) imaging;
(b) constructing a digital model for characterizing the tooth replica by segmenting selected images of the tooth replica;
(c) converting the selected segmented images to stereo lithography (STL) images;
(d) transmitting the STL files of the tooth replica to a computer aided manufacturing process for fabricating the tooth replica;
(e) selecting and transmitting STL files of the customized socket preparation tool to a computer aided manufacturing process for fabricating the socket preparation tool;
(f) selecting the socket preparation tool and coupling the socket preparation tool to the tooth replica;
(g) fitting and adjusting the socket preparation tool coupled to the tooth replica in the recipient site; and
(h) after step (g), extracting the donor tooth.

2. The method of claim 1, wherein steps (d) and (e) comprise milling the tooth replica or the socket preparation tool from unsintered zirconium block.

3. The method of claim 1, further comprises step (i) driving the socket forming tool in at least one direction via a driver configured to be directly or indirectly coupled to the socket forming tool.

4. The method of claim 3, wherein the driver is configured to be directly or indirectly coupled to a receiver of the socket forming tool.

5. The method of claim 1, further comprising step (j) fitting the donor tooth in the recipient site.

6. The method of claim 1, wherein the customized socket preparation tool is selected to accommodate a form factor of the tooth replica or a spectrum of form factors of the tooth replica within an expected range of values based on the donor tooth.

7. A socket preparation tool configured to facilitate autotransplantation of a donor tooth to a recipient site, the tool comprising:
a tooth replica of the donor tooth;
a coronal portion for facilitating alignment of the tooth replica adjacent teeth during a simulated autotransplantation;
a radicular portion comprising a smooth surface configured to facilitate compression of soft bone of the tooth replica during a simulated autotransplantation; and
a cylindrical attachment configured to be coupled to the tooth replica, the attachment comprising a receiver.

8. The tool of claim 7, wherein the receiver is configured to be directly or indirectly coupled to a driver for driving the socket forming tool in at least one direction in order to adjust the fit of the socket preparation tool in the recipient site.

9. The tool of claim 7, wherein the socket preparation tool is customized to accommodate a form factor of the tooth replica or a spectrum of form factors of the tooth replica within an expected range of value based on the donor tooth.

10. The tool of claim 9, wherein the radicular portion of the socket preparation tool is adjusted to accommodate the form factor or the spectrum of form factors.

11. A socket preparation tool configured to facilitate autotransplantation of a donor tooth to a recipient site, the tool comprising:
a tooth replica of the donor tooth;
a coronal portion for facilitating alignment of the tooth replica adjacent teeth during a simulated autotransplantation;
a radicular portion comprising a contoured surface adapted for filing bone of the tooth replica during a simulated autotransplantation; and
a cylindrical attachment configured to be coupled to the tooth replica, the attachment comprising a receiver.

12. The tool of claim 11, wherein the receiver is configured to be directly or indirectly coupled to a driver for driving the socket forming tool in at least one direction in order to adjust the fit of the socket preparation tool in the recipient site.

13. The tool of claim 12, wherein the socket preparation tool is customized to accommodate a form factor of the tooth replica or a spectrum of form factors of the tooth replica within an expected range of value based on the donor tooth.

14. The tool of claim 13, wherein the radicular portion of the socket preparation tool is adjusted to accommodate the form factor or the spectrum of form factors.

15. A socket preparation tool configured to facilitate autotransplantation of a donor tooth to a recipient site, the tool comprising:
a tooth replica of the donor tooth;
a radicular portion comprising a smooth surface configured to facilitate compression of soft tissue during a simulated autotransplantation; and
a cylindrical attachment configured to be coupled to the tooth replica, the attachment comprising a receiver.

16. The tool of claim 15, wherein the receiver is configured to be directly or indirectly coupled to a driver for driving the socket forming tool in at least one direction in order to adjust the fit of the socket preparation tool in the recipient site.

17. A socket preparation tool configured to facilitate autotransplantation of a donor tooth to a recipient site, the tool comprising:
a tooth replica of the donor tooth;
a radicular portion comprising a contoured surface adapted for cutting bone during a simulated autotransplantation; and
a cylindrical attachment configured to be coupled to the tooth replica, the attachment comprising a receiver.

18. The tool of claim 17, wherein the receiver is configured to be directly or indirectly coupled to a driver for driving the socket forming tool in at least one direction in order to adjust the fit of the socket preparation tool in the recipient site.

19. Socket preparation tools configured to facilitate autotransplantation of a donor tooth to a recipient site,
a first tool comprising:
a tooth replica of the donor tooth;
a radicular portion comprising a smooth surface configured to facilitate compression of soft tissue during a simulated autotransplantation; and
a cylindrical attachment configured to be coupled to the tooth replica, the attachment comprising a respective receiver;
the second tool comprising:
a radicular portion comprising a contoured surface adapted for cutting bone during the simulated autotransplantation; and
a cylindrical attachment configured to be coupled to the tooth replica, the attachment comprising a respective receiver.

20. A system for guided autotransplantation of a donor tooth comprising the socket preparation tools of claim 19.
